(19) United States
(12) Patent Application Publication
(10) Pub. No.: US 2019/0091056 A1
(43) Pub. Date: Mar. 28, 2019

(54) POSTURE CORRECTING DEVICE

(71) Applicant: Jae Woo LEE, Seongnam-si (KR)

(72) Inventor: Jae Woo LEE, Seongnam-si (KR)

(21) Appl. No.: 16/059,404

(22) Filed: Aug. 9, 2018

(30) Foreign Application Priority Data
Sep. 28, 2017 (KR) 10-2017-0126172

(51) Int. Cl. A61F 5/02; A61H 1/02; A63B 23/02

(52) U.S. Cl. CPC A61F 5/02; A61H 2203/0425; A63B 23/02; A61H 1/0292

(57) ABSTRACT

A posture correcting device is provided. The posture correcting device includes: armpit rests bent forward to support both armpits of a user; vertical rods each of which extends downward from each of the armpit rests; and lower clothing supports each of which is formed at the lowermost end of each of the vertical rods and fixed to a lower clothing piece of the user.

### CROSS-REFERENCE TO RELATED APPLICATION(S)

This application claims the benefit under 35 USC § 119(a) of Korean Patent Application No. 10-2017-0126172, filed on Sep. 28, 2017, in the Korean Intellectual Property Office, the entire disclosure of which is incorporated herein by reference for all purposes.

### BACKGROUND

### 1. Field

The following description relates to a posture correcting device.

### 2. Description of Related Art

Recently, there are increasing types of jobs in which workers process documents while leaning over a desk or carry out tasks for long hours using a computer. Accordingly, the number of people with various body-shape-related medical conditions due to abnormal curvature of the spine or spinal deformity which may occur as a result of long hours of computer or paper works is rapidly increasing.

Keeping a right posture at all times during work and doing exercise consistently may help to maintain a correct posture and prevent such spinal deformity, but it is quite difficult for ordinary people living busy lives to follow such instructions.

In particular, spinal diseases, such as scoliosis, occur even in younger students. If such a spinal disease continues to develop, it can cause a pain in the body even in a sitting position, resulting in a decrease in concentration and memory, as well as lowering of work efficiency.

In order to correct the deformed spine, a band-type spine support made of mostly an elastic material and a spine support made of rigid plastic are used. However, such conventional spine supports have drawbacks that they are inconvenient to put on and take off and difficult to continuously use in daily life.

Therefore, there is a continuing need for development of a posture correcting device which is simple in structure and capable of being easily worn and removed in daily life.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

The following description relates to a posture correcting device which is simple in structure and easy to use in daily life.

According to one embodiment, there is provided a posture correcting device including: armpit rests bent forward to support both armpits of a user; vertical rods each of which extends downward from each of the armpit rests; and lower clothing supports each of which is formed at the lowermost end of each of the vertical rods and fixed to a lower clothing piece of the user.

The vertical rods may include a left vertical rod supporting a left part of the user and a right vertical rod supporting a right part of the user and the posture correcting device further may comprise a horizontal rod connecting the left vertical rod and the right vertical rod.

The lower clothing supports may include a left lower clothing support formed at the lowermost end of the left vertical rod and a right lower clothing support formed at the lowermost end of the right vertical rod.

The lower clothing supports may be formed in a A-shape or a ∩-shape having an opening facing downward.

The posture correcting device further may comprise padded cushions each of which is formed of at least one of rubber, sponge, bondage, and cotton on a surface of each of the armpit rests.

The posture correcting device further may comprise height adjusting portions each of which is detachable from a middle of each of the vertical rods.

The posture correcting device further may comprise an upper garment covering a surface of the posture correcting device.

Other features and aspects will be apparent from the following detailed description, the drawings, and the claims.

### BRIEF DESCRIPTION OF THE DRAWINGS

FIG. 1 is a perspective view illustrating a posture correcting device according to one embodiment of the present invention.

FIG. 2 is a perspective view illustrating a posture correcting device according to another embodiment of the present invention.

FIG. 3 is a perspective view illustrating a posture correcting device according to still another embodiment of the present invention.

FIG. 4 is a diagram schematically illustrating a state in which a user is wearing a posture correcting device according to one embodiment of the present invention.

Throughout the drawings and the detailed description, unless otherwise described, the same drawing reference numerals will be understood to refer to the same elements, features, and structures. The relative size and depiction of these elements may be exaggerated for clarity, illustration, and convenience.

### DETAILED DESCRIPTION

Hereinafter, embodiments of the present invention will be described in detail with reference to the accompanying drawings so as to be easily practiced by a person of ordinary skill in the art. It should be understood that the present invention is not to be construed as limited to the embodiments set forth herein and may be embodied in many different forms.

Any redundant descriptions of well-known parts will be omitted for clarity, and like reference numerals refer to like elements throughout the specification.

Sizes and thicknesses of elements in the drawings may be exaggerated for convenience of description. In other words, since sizes and thicknesses of components in the drawings are arbitrarily illustrated for convenience of description, the following embodiments are not limited thereto. In the drawings, thicknesses are enlarged for clarity of various layers and regions, and thicknesses of some layers and regions are exaggerated for convenience of description.

In the specification, unless explicitly described to the contrary, the word “comprise” and variations such as “comprises” or “comprising,” will be understood to imply the inclusion of stated elements but not the exclusion of any other elements.

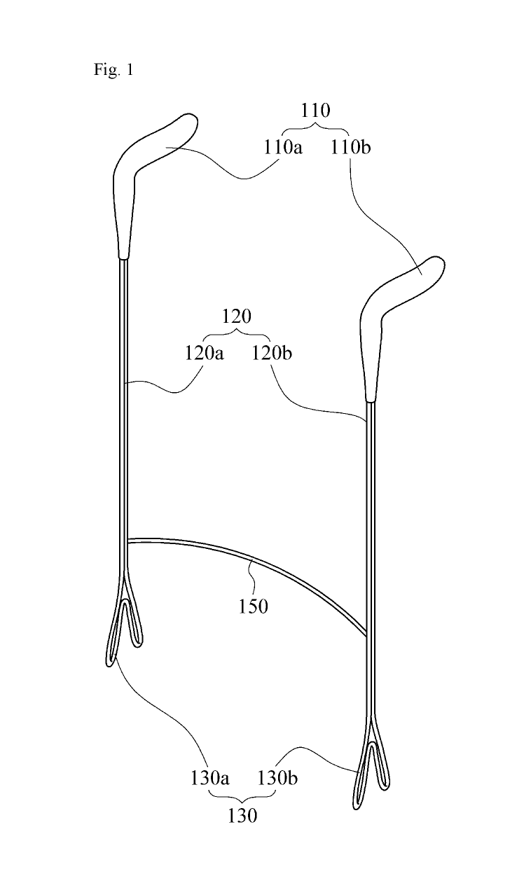

Hereinafter, a posture correcting device according to one embodiment of the present invention will be described in detail with reference to FIG. 1.

FIG. 1 is a perspective view illustrating a posture correcting device according to one embodiment of the present invention.

Referring to FIG. 1, the posture correcting device according to one embodiment of the present invention includes two armpit rests 110 to support both armpits of a user, two vertical rods 120 extending downward from the armpit rests 110, lower clothing supports 130 formed at the lowermost ends of the vertical rods 120, and a horizontal rod 150 connected between the two spaced vertical rods 120 to support both vertical rods 120.

The armpit rests 110 of the posture correcting device according to one embodiment of the present invention include a left armpit rest 110a to support the left armpit of the user and a right armpit rest 110b to support the right armpit of the user, and support the weight of both armpits to hold up the user's torso.

That is, the armpit rests 110 may reduce the weight of both arms and torso exerted on the spine by providing a lifting force to muscles of areas where the user's shoulder blades and the arms (upper limb) are connected to the torso.

The armpit rests 110 may be formed to be inclined forward at a predetermined angle with respect to the vertical rods 120 of the posture correcting device so as to support both armpits of the user.

Since the armpit rests 110 support the weight of the user's torso when worn by the user, each of the armpit rests 110 may further include a padded cushion of a soft material on a surface thereof.

The padded cushion may be formed of a material, such as rubber, sponge, a bandage, cotton, or the like, but the material is not limited thereto, so long as it can provide a soft texture.

The vertical rods 120 extending downward from the armpit rests 110 include a left vertical rod 120a extending from the left armpit rest 110a and a right vertical rod 120b extending from the right armpit rest 110b, and serve to connect the armpit rests 110 and the lower clothing supports 130 described below.

The vertical rods 120 may be formed to be height adjustable as needed by including a known structure for adjusting a height thereof according to the height of the user.

Then, the lower clothing supports 130 formed at the lowermost ends of the vertical rods 120 include a left lower clothing support 130a formed at the lowermost end of the left vertical rod 120a and a right lower clothing support 130b formed at the lowermost end of the right vertical rod 120b. The lower clothing supports 130 are fixed to a rear potion of a lower clothing piece of the user and serve to secure the lower part of the posture correcting device to the lower clothing piece of the user.

The lower clothing supports 130 may be A-shaped or ∩-shaped having an opening facing downward to be fixed to the lower clothing piece of the user. That is, a waist part of the user's lower clothing piece may be inserted and fixed to the opening of the A-shaped or ∩-shaped lower clothing supports 130.

Additionally, the lower clothing supports 130 may be fixed to the lower clothing piece, such as pants, a skirt, or the like, which the user is wearing, and may be fixed to a belt on the pants or skirt.

The lower clothing supports 130 are fixed to a rear portion of the user's lower clothing piece and transmit the weight exerted by the armpit rests 110 to the rear portion of the lower clothing piece so that a pressure may be applied to the rear portion of the lower clothing piece, and this pressure may be transmitted to a front portion of the lower clothing piece and applied to the user's glutei maximi, iliac crest and abdominal muscles.

The pressure transmitted by the lower clothing supports 130 may stimulate the gluteus maximi, iliac crest and the abdominal muscles, thereby promoting the development of muscles around the user's waist, which may help the vertebra to place in correct position or help to relieve back pain.

The horizontal rod 150 formed between the two spaced vertical rods 120 serves to connect and support the left vertical rod 120a and the right vertical rod 120, which are spaced apart from each other. For convenience of illustration, only one horizontal rod 150 is illustrated, but a plurality of horizontal rods 150 may be formed as needed.

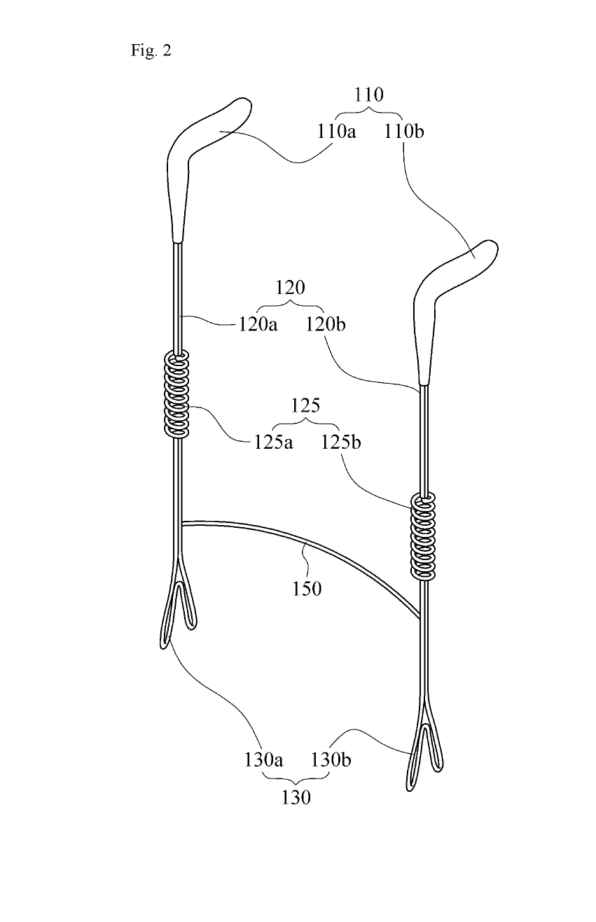

Hereinafter, a posture correcting apparatus according to another embodiment of the present invention will be described in detail with reference to FIG. 2.

FIG. 2 is a perspective view illustrating a posture correcting device according to another embodiment of the present invention.

The posture correcting device shown in FIG. 2 according to another embodiment of the present invention is substantially the same as the posture correcting device according to the embodiment of FIG. 1, except for height adjusting portions 125, and hence redundant descriptions will be omitted.

Referring to FIG. 2, the posture correcting device according to another embodiment of the present invention may further include height adjusting portions 125 each detachable from a middle of a respective one of vertical rods 120.

The height adjusting portions 125 may include a left height adjusting portion 125a for adjusting a height of a left vertical rod 110a and a right height adjusting portion 125b for adjusting a height of a right vertical rod 110b.

Each of the vertical rods 120 of the posture correcting device may be separated in the middle in order to detach the height adjusting portion 125 therefrom, and the height adjusting portion 125 is mounted between the separated parts of the vertical rod 120, so that the height of the posture correcting device can be adjusted.

The height of the height adjusting portions 125 can be adjusted appropriately according to the height of the user and may be adjusted to a length suitable for various users' body sizes.

Each of the height adjusting portions 125 may be in the form of a spring or a stick.

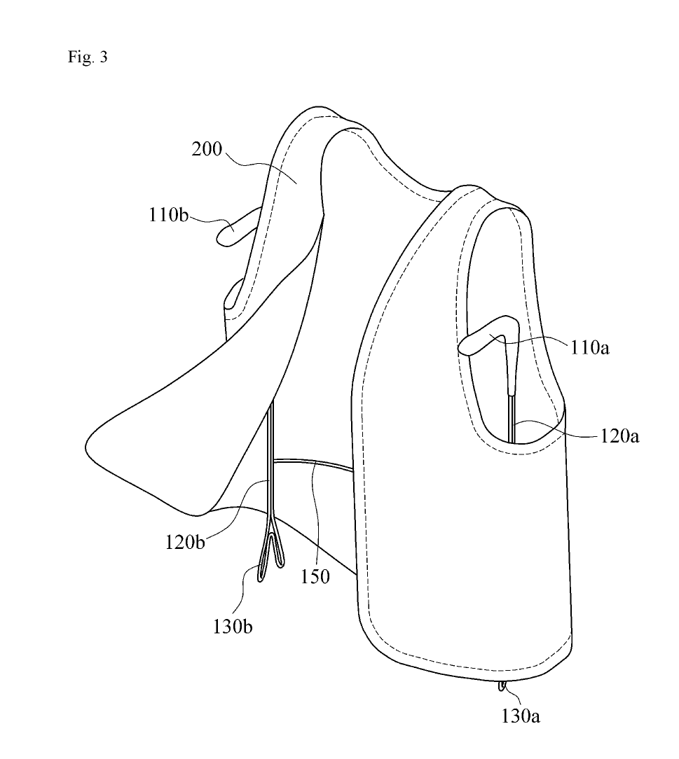

Then, a posture correcting device according to still another embodiment of the present invention will be described with reference to FIG. 3.

FIG. 3 is a perspective view illustrating a posture correcting device according to still another embodiment of the present invention.

The posture correcting device shown in FIG. 3 according to still another embodiment of the present invention is substantially the same as the posture correcting device according to the embodiment of FIG. 1 except for an upper garment attached to a surface of the posture correcting device, and hence redundant descriptions will be omitted.

Referring to FIG. 3, the posture correcting device according to still another embodiment of the present invention may have an upper garment attached to a surface thereof.

The upper garment may include a jacket, a vest, and the like, and the posture correcting device is attached to the inside of the upper garment, so that the user can continuously correct the posture through the posture correcting device even by only wearing the upper garment during daily life.

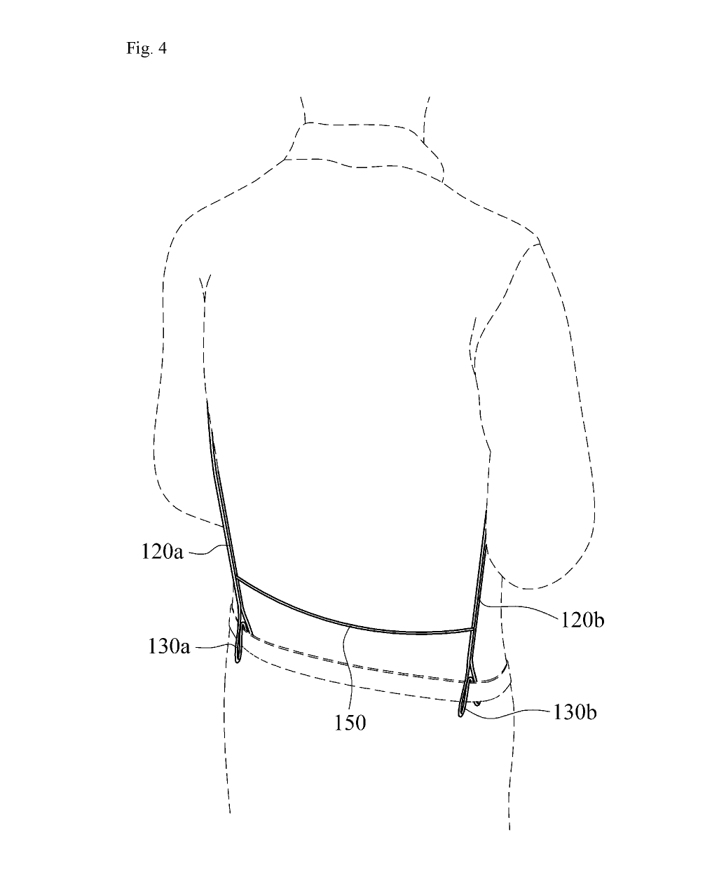

Then, a state in which a posture correcting device according to one embodiment of the present invention is worn will be described in brief with reference to FIG. 4.

FIG. 4 is a diagram schematically illustrating a state in which a user is wearing a posture correcting device according to one embodiment of the present invention.

As shown in FIG. 4, armpit rests 110 of the posture correcting device according to one embodiment of the present invention may be fixedly positioned under the armpit areas of a user and support the armpits and lower clothing supports 130 formed at the lowermost ends of vertical rods 120 extending downward from the respective armpit rests 110 may be fixed to the user's lower clothing piece.

That is, as shown in FIG. 4, the posture correcting device according to one embodiment of the present invention may support the weight of the user's torso and thereby distribute the weight of the torso applied to the vertebrae to the user's glutei maximi, iliac crest and abdominal muscles, so that the spine can brought in correct shape and the development of muscles around the waist can be promoted, which helps the spine to be in correct position or helps to relieve back pain.

As described above, the posture correcting device according to the embodiments of the present invention has a simple structure and is easy to use in daily life.

A number of examples have been described above. Nevertheless, it will be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

What is claimed is:

1. A posture correcting device comprising:
armpit rests bent forward to support both armpits of a user;
vertical rods each of which extends downward from each of the armpit rests; and
lower clothing supports each of which is formed at the lowermost end of each of the vertical rods and fixed to a lower clothing piece of the user.

2. The posture correcting device of claim 1, wherein the vertical rods include a left vertical rod supporting a left part of the user and a right vertical rod supporting a right part of the user and the posture correcting device further comprises a horizontal rod connecting the left vertical rod and the right vertical rod.

3. The posture correcting device of claim 2, wherein the lower clothing supports include a left lower clothing support formed at the lowermost end of the left vertical rod and a right lower clothing support formed at the lowermost end of the right vertical rod.

4. The posture correcting device of claim 3, wherein the lower clothing supports are formed in a A-shape or a ∩-shape having an opening facing downward.

5. The posture correcting device of claim 1, further comprising padded cushions each of which is formed of at least one of rubber, sponge, bondage, and cotton on a surface of each of the armpit rests.

6. The posture correcting device of claim 1, further comprising height adjusting portions each of which is detachable from a middle of each of the vertical rods.

7. The posture correcting device of claim 1, further comprising an upper garment covering a surface of the posture correcting device.
